SEQUENCE LISTING (18 sequences)

SEQ ID NO: 1 - DNA, 21 bp, Artificial Sequence
  Description of Artificial Sequence Synthetic primer
tctcgtagtctgctttgctca

SEQ ID NO: 2 - DNA, 20 bp, Artificial Sequence
  Description of Artificial Sequence Synthetic primer
ctcatctttcacctcacccc

SEQ ID NO: 3 - DNA, 16 bp, Artificial Sequence
  Description of Artificial Sequence Synthetic primer
ctcaggcgtcaggctc

SEQ ID NO: 4 - DNA, 22 bp, Artificial Sequence
  Description of Artificial Sequence Synthetic primer
ctgcactcaataaaccctcaat

SEQ ID NO: 5 - DNA, 10 bp, Artificial Sequence
  Description of Artificial Sequence Synthetic probe
  c-term amidated
cctttttccc

SEQ ID NO: 6 - DNA, 10 bp, Artificial Sequence
  Description of Artificial Sequence Synthetic probe
  c-term amidated
cctcctctct

SEQ ID NO: 7 - DNA, 21 bp, Artificial Sequence
  Description of Artificial Sequence Synthetic primer
tgagcaaagcagactacgaga

SEQ ID NO: 8 - DNA, 20 bp, Artificial Sequence
  Description of Artificial Sequence Synthetic primer
ggggtgaggtgaaagatgag

SEQ ID NO: 9 - DNA, 16 bp, Artificial Sequence
  Description of Artificial Sequence Synthetic primer
gagcctgacgcctgag

SEQ ID NO: 10 - DNA, 22 bp, Artificial Sequence
  Description of Artificial Sequence Synthetic primer
attgagggtttattgagtgcag

SEQ ID NO: 11 - DNA, 21 bp, Artificial Sequence
  Description of Artificial Sequence Synthetic primer
gcattccgctgaccatcaata

SEQ ID NO: 12 - DNA, 18 bp, Artificial Sequence
  Description of Artificial Sequence Synthetic primer
tccaacgagcggcttcac

SEQ ID NO: 13 - DNA, 22 bp, Artificial Sequence
  Description of Artificial Sequence Synthetic primer
ccactggattagcagagttcaa

SEQ ID NO: 14 - DNA, 25 bp, Artificial Sequence
  Description of Artificial Sequence Synthetic primer
cagcggccagtagcatctgctttga

SEQ ID NO: 15 - DNA, 19 bp, Artificial Sequence
  Description of Artificial Sequence Synthetic primer
caactgggacgactggaga

SEQ ID NO: 16 - DNA, 20 bp, Artificial Sequence
  Description of Artificial Sequence Synthetic primer
gaagatggtgatgggatttc

SEQ ID NO: 17 - DNA, 20 bp, Artificial Sequence
  Description of Artificial Sequence Synthetic primer
caagcttcccgttctcagcc

SEQ ID NO: 18 - DNA, 20 bp, Artificial Sequence
  Description of Artificial Sequence Synthetic primer
caagcttcccgttctcagcc

(19) United States
(12) Patent Application Publication
(10) Pub. No.: US 2007/0184470 A1
(43) Pub. Date: Aug. 9, 2007

(54) METHOD TO MEASURE GENE EXPRESSION RATIO OF KEY GENES

(76) Inventors: Pierre Aman, Bjorkvagen (SE); Anders Stalberg, Ranunkelgatan (SE); Mikael Kubista, Nedre Solstensvagen (SE)

Correspondence Address: GAUTHIER & CONNORS, LLP, 225 FRANKLIN STREET, SUITE 2300, BOSTON, MA, 02110, US

(21) Appl. No.: 11/617,214

(22) Filed: Dec. 28, 2006

Related U.S. Application Data
(62) Division of application No. 10/694,979, filed on Oct. 28, 2003, now abandoned.
Application No. 10/694,979 is a continuation of application No. PCT/SE02/01093, filed on Jun. 5, 2002.

(30) Foreign Application Priority Data
Jun. 6, 2001 (SE) 0101999-1
Nov. 27, 2001 (SE) 0103991-6

(51) Int. Cl. C12Q 1/68; G06F 19/00; C12P 19/34

(52) U.S. Cl. 435/6; 435/91.2; 702/20

(57) ABSTRACT

The invention is a method to determine the amounts, in particular the relative amounts, of nucleic acids in complex biological samples by means of real-time PCR. According to the invention the biological sample is systematically diluted and each dilution is studied by real-time PCR for all genes of interest. From the dependence of the threshold cycle on dilution factor for each of the genes, the PCR efficiencies of the reactions are determined in the particular samples. Determining also the relative sensitivity of the real-time PCR assays compared, the relative amounts of two nucleic acids in complex biological samples are determined with unprecedented accuracy.

### TECHNICAL FIELD

The invention belongs to the category methods for quantification of nucleic acids. Such methods are used to determine the amount of specific genes, gene segments, RNA molecules and other nucleic acids in samples. These methods are primarily used in clinical diagnosis, for example, to test tissue, blood and urine samples, and in food technology, agriculture and biomedicine.

### BACKGROUND OF THE INVENTION

Methods to measure gene expression go back to the 1970s. The first method was based on measuring reassociation kinetics of complementary strands (Wetmur & Davidson, J. Mol. Biol., 1, 349, 1968). A radiolabeled single-stranded DNA probe was added and its association with complementary mRNA, when the mRNA was present in molar excess, was measured. These were very difficult experiments, for several reasons: the concentrations of reagents in the hybridization reactions were often so low that the reassociation reaction required many hours—days in some cases—to generate significant amounts of hybrid. Second, the hydroxyapatite columns routinely used to separate double-stranded and single-stranded nucleic acids were messy to work with, which made the whole procedure tedious.

10 years later Northern hybridization was developed (Alwine, Kemp, & Stark, Proc. Natl. Acad. Sci. U.S.A. 74, 5350, 1977). Here the RNA was immobilized on cellulose and later nitrocellulose paper to which radiolabeled probes were hybridized. The method has several disadvantages. Its capacity to bind nucleic acids is low and varies according to the size of the RNA. In particular, nucleic acids<400 bases in length are retained inefficiently. Since the RNA is attached to the nitrocellulose by hydrophobic interaction, rather than covalently, it leaches slowly from the matrix during hybridization and washing at high temperatures.

Ribonuclease protection assay (Pape, Melchior, & Marotti, Genet. Anal. Tech. Appl. 8, 206, 1991) is 20-100 fold more sensitive than northern hybridization being capable of detecting about 10⁵ copies of a specific transcript. It can cope with several target mRNAs simultaneously and, because the intensity of the signal is directly proportional to the concentration of target RNA, comparison of the level of expression of the target gene in different tissues is easily accomplished. A disadvantage is that it works best with antisense probes that are exactly complementary to the target RNA, which is a problem if the experiment generates RNA-RNA hybrids containing mismatched base pairs that are susceptible to cleavage by RNase, for example, when analyzing families of related mRNAs.

In 1983 the polymerase chain reaction (PCR) to amplify nucleic acids in an exponential process was invented (U.S. Pat. No. 4,683,202). This opened the possibility to quantify even minute amounts of a nucleic acid in a sample. In traditional PCR the DNA (or RNA after conversion to cDNA) in the sample was amplified first and then detected in a separate step. This made quantification very uncertain, since the reaction usually ran short of some components giving rise to the same amount of product irrespectively of the amount of starting template.

This problem was solved by inventing real-time PCR (U.S. Pat. No. 6,171,785), where fluorescent dyes or fluorescent probes (N. Svanvik, G. Westman, W. Dongyuan & M. Kubista. Anal. Biochem. 281, 26-35, 2000) are included in the reaction to provide for real-time monitoring of the product formed. The number of amplification cycles required to reach a particular signal threshold level, number of amplification cycles at threshold (CT), is registered. Traditionally the number of template copies in the test sample is estimated by comparing the measured CT value with CT values measured for standard samples containing known amounts of template. This approach is highly accurate when the test sample is of similar complexity as the standard samples, which usually are dilutions of plasmid or purified DNA template. This relies on the crucial assumption that PCR efficiencies in test and standard samples are the same. If this is not the case a CT-value measured in a test sample will correspond to a different number of cDNA copies then the same CT-value measured in the standard sample. The error introduced by such assumption may be substantial owing to accumulation effects. For example, 80% efficiency in the test sample and 85% efficiency in the standard sample results in 50% difference in the number of DNA copies after 25 cycles (eq. 1).
N_{CT}═N₀*(1+E)^{CT}

The common method to account for differences in PCR efficiencies between test and standard samples is to amplify a reference gene, usually a housekeeping gene, in parallel and relating the expression of the studied target gene to the expression of the housekeeping gene. This, of course, relies on the assumption that the expression level of the housekeeping gene is constant among the samples being compared, which has been questioned (Bustin S A: Absolute quantification of mRNA using real-time reverse transcription polymerase chain reaction assays. J Mol Endocrinol 2000, 25:169-193; Suzuki T, Higgins P J, Crawford D R: Control Selecton for RNA Quantitation. BioTechniques 2000, 29:332-337; Schmittgen T D, Zakrajsek B A: Effect of experimental treatment on housekeeping gene expression: validation by real-time, quantitative RT-PCR. J Biochem Biophys Methods 2000, 46:69-81). Further, which is rarely acknowledged, it also assumes that the efficiencies of the two reactions, i.e., the PCR of the target gene and the PCR of the housekeeping gene, are inhibited to the same degree in the standard sample as well as in the test sample (eq. 2):

$$\frac{( 1 + {E}_{target gene}^{test sample} )}{( 1 + {E}_{housekeeping gene}^{test sample} )} = \frac{( 1 + {E}_{target gene}^{standard sample} )}{( 1 + {E}_{housekeeping gene}^{standard sample} )}$$

The validity of this critical assumption has not been tested, because there has been no method to determine the PCR efficiencies of individual reactions in samples.

One object of the present invention is to overcome the limitations discussed above with traditional methods to determine gene expression and also the limitations of the present real-time PCR approach to quantify the relative amounts of two nucleic acids in a biological sample.

Another object of the present invention is to diagnose a disease, such as cancers and in particular lymphomas, with very high sensitivity by measuring the ratio of expression of key genes.

Still another object of the present invention is to diagnose a disease with technology that requires very little material as obtained, for example, with fine needle aspiration biopsy.

Still another object of the present invention is to make diagnosis rapid and more cost efficient.

### DESCRIPTION OF FIGURES

FIG. 1. Controlled dilution of test sample. The test sample is diluted 64 times in three steps á four times.

FIG. 2. Inter and intra assays. Top left: IgLκ intra assay; top right: IgLλ intra assay; bottom left: IgLκ inter assay; bottom right: IgLλ inter assay.

FIG. 3. Variations in inter and intra assays. Variations in CT-values for the IgLκ and IgLλ reactions in eight repeated measurements run either in parallel (intra-assay) or separately (inter-assay) of sample BR0.

FIG. 4. PCR efficiencies of the IgLκ (A) and IgLλ (B) assays. The lines are normalized at maximum template concentration. PCR efficiencies are obtained from to the slopes of the fitted lines as E=10^{-(slope)}^{^{−1}}−1. The outlier, sample BR17, is indicated with dotted line ( . . . ). Purified template is shown with dashed line ( - - - ). For all lines R²>0.99.

FIG. 5. IgLκ and IgLλ PCR efficiencies in lymphoma samples. PCR efficiencies of the IgLκ and IgLλ reactions determined by the invented approach in seven test samples and of purified template. The calculated relative sensitivity, K_{RS}, in the negative samples is also shown.

FIG. 6. Classification of lymphoma samples. Patient samples shown in a CTκ vs. CTλ plot. Each symbol represents one sample and is depicted at its CTκ and CTλ values. The opposite axes indicate the number of cDNA copies for purified template. The straight solid line represents (CTκ, CTλ) values expected for negative samples calculated assuming 85.4% and 79.3% PCR efficiencies for the IgLκ and the IgLλ reactions, respectively. The dotted lines ( . . . ) indicate an interval within which negative samples should be found with at least 95% probability. B-cell lymphomas are shown with ▪, diffuse large B-cell lymphoma with * and negative samples with ●. Open symbols indicate corrected CT-values of samples for which specific PCR efficiencies were determined.

FIG. 7. Comparison of classification by various methods of NHL samples. Classification of patient samples by the invented real-time PCR method compared with traditional R.E.A.L. classification, classification by IHC clonality and by flow cytometry. Positive B cell lymphoma samples are shown in bold. The more rapid and for the patient less inconvenient invented real-time PCR method does in all cases agree with the traditional methods.

FIG. 8. Determination of PCR efficiencies for bcr-abl and GAPDH using probes. CT values measured for the bcr-abl and GAPDH reactions using Taqman probe real-time PCR assays in a patient sample systematically diluted in steps of two. The CT v.s. log (dilution) plots have different slopes evidencing that the two reactions are inhibited to different degrees in the sample. The ratio between bcr-abl and GAPDH cDNA are calculated taking the CR efficiencies into account.

FIG. 9. PCR efficiencies of bcr-abl and GAPDH reactions in patient samples. Table showing the PCR efficiencies of the bcr-abl and GAPDH reactions measured using Taqman probe real-time PCR assays in five patient samples determined by the invented method. In all samples was the GAPDH reaction inhibited to a higher degree. The degree of inhibition of both reactions also vary substantially among the samples evidencing the importance of the present invention.

FIG. 10. Determination of bcr-abl cDNA using dye. Real-time PCR amplification curves of a SYBRGreen assay of bcr-abl cDNA. Top left shows plot of CT v.s. log (starting concentration) and top right shows melting curves distinguishing template specific products from primer dimers.

FIG. 11. Determination of GAPDH cDNA using dye. Real-time PCR amplification curves of a SYBRGreen assay of GAPDH cDNA. Top left shows plot of CT versus log (starting concentration) and top right shows melting curves distinguishing template specific products from primer dimers.

### SUMMARY OF THE PRESENT INVENTION

The present invention is a method to determine the relative amounts of two nucleic acids, in particular two cDNAs, in complex biological samples by real-time PCR. It is based on determining the threshold cycles (CT) of the PCR:s of a dilution series of the test sample, and from the dependences of CT on the logarithm of the dilution factor determine the PCR efficiencies of the two reactions in the particular sample.

With the here invented method it is possible to determine PCR efficiency in biological test samples.

With the here invented method it is possible to determine the ratio of two nucleic acids in biological test samples with unprecedented accuracy by taking into account the sample specific inhibition.

With the here invented method it is possible to determine the ratio of two cDNA and thereby indirectly of the corresponding mRNAs and, hence, the relative expression of two genes.

With the here invented method it is possible to determine the ratio of the expression of IgLκ and IgLλ genes thereby detecting clonality of B cells and classifying lymphoma.

The fundamental inventive idea is that the sample itself is used as a standard reference by using a dilution or a concentrate thereof as comparative standard.

### DETAILED DESCRIPTION OF THE INVENTION AND ITS PREFERRED EMBODIMENTS

As indicated by the title, the present invention is a procedure to determine the ratio of two nucleic acids, in particular of two cDNAs and hence mRNAs, in complex biological samples by quantitative real-time PCR. As already mentioned the state-of-the art approach expresses the amount of a nucleic acid in a sample relative to the amount of another nucleic acid. This is the typical case both when measuring viral loads as well as gene expression levels. Typically the expression of the gene of interest is expressed relative to the expression of a house keeping gene, which is a gene assumed to be expressed to the same degree under essentially all conditions. This relative expression of two genes relies on the assumption that the two PCR:s are inhibited to the same degree in the standard sample as well as in the test sample (eq. 0). So far it has not been possible to test this assumption, because there has been no way to determine PCR efficiencies in individual samples. This is made possible with the invention described here.

Although one might be inclined to think that inhibitory components that may be present in biological samples should have the same effect on all PCR:s, it may not necessarily be so. The degree of inhibition may depend on features that are particular for the different PCR systems, such as the length and sequence of template, template tertiary structure, lengths and sequences of primers etc. Inhibition may also be indirect through competition for critical elements such as ions and dNTPs. If two PCR systems have optimum efficiencies at different concentrations of Mg²⁺, dNTP, primers and dye/probe elements in biological samples that interact with these PCR components may interfere with the reactions to different degrees.

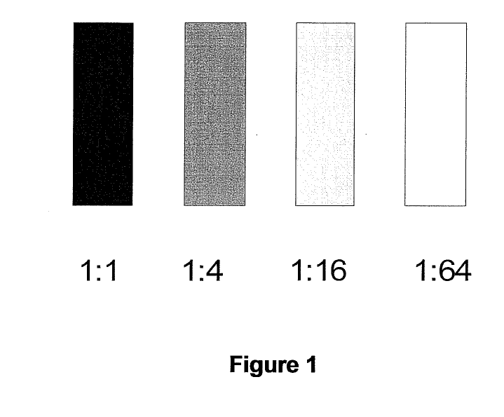

The invented approach is based on taking the test sample and performing a controlled dilution, for example, as illustrated in FIG. 1, in four steps a four times. By amplifying the nucleic acid in each of these dilutions and comparing the number of cycles required to reach threshold (CT) with-the dilution factors, the efficiency of the PCR in that particular sample can be determined. For example, if the reaction proceeds with 100% efficiency, 4 times dilution should increase the CT exactly by 2, 16 times dilution by four and 64 times dilution by 8. From a plot of CT vs. log (dilution factor) the efficiency of the reaction in that particular sample is determined. When comparing the expression of two genes in a biological test sample, the test sample (after cDNA synthesis) is serially diluted and the amounts of both cDNAs are determined in each dilution, from which the PCR efficiencies of both reactions in that particular sample are determined.

A mathematical model is developed to determine the ratio of the expression levels of two genes by real-time PCR. The model is general and applied here on the IgLκ and IgLλ genes. In the following equations the following meanings are due:

N_{0A} means the number of units, N_{A}, at the time 0 of cDNA of type A

N_{0B} means the number of units, N_{B}, at the time 0 of cDNA of type B

K_{RS} means the constant based on relative sensitivity for optical detection

E_{A} means PCR efficiency of sample A

E_{B} means PCR efficiency of sample B

[E_{A}] means PCR mean efficiency determined on a larger number of samples of A

[E_{B}] means PCR mean efficiency determined on a larger number of samples of B

CT_{A} means the number of cycles of amplifications in reaction of sample A to reach threshold value.

CT_{B} means the number of cycles of amplifications in reaction of sample B to reach threshold value.

The basic equation describing real-time PCR amplification in exponential phase is (eq. 3):
N_{CT}═N₀*(1+E)^{CT}

N₀ is the number of cDNA molecules, E is the PCR efficiency (E=1 corresponds to 100% efficiency and is expressed in percentage throughout), CT is the threshold cycle and N_{CT} is the number of template copies present after CT PCR cycles. E is assumed to be independent of N in the particular amplification range. It is determined by performing a dilution series of mRNA or cDNA standard and is calculated from the slope in a CT vs. log N₀ plot (eq. 4):
E=10^{-(slope)}^{^{−1}}−1

The fluorescence increase, i.e., the fluorescence signal after subtraction of background, at threshold is proportional to the amount of target DNA (eq. 5):
I=k*N_{CT}

k is a system and instrument constant and N_{CT} is the number of target DNA molecules pre-sent at threshold. The relative expression of the IgLκ and IgLλ genes is obtained as (eq. 6, eq. 7, eq. 8, and eq. 9)
N_{CT}_{_{IgLκ}}═N₀_{_{IgLκ}}*(1+E_{IgLκ})^{CT}^{_{IgLκ}}
I_{IgLκ}=k_{IgLκ}*N_{CT}_{_{IgLκ}}
N_{CT}_{_{IgLλ}}═N₀_{_{IgLλ}}*(1+E_{IgLλ})^{CT}^{_{IgLλ}}
I_{IgLλ}=k_{IgLλ}*N_{CT}_{_{IgLλ}}

At threshold I_{IgLκ}═I_{IgLλ}. Equating eq. 5 with eq.7 and rearranging we obtain (eq. 10):

$${K}_{RS} = \frac{{k}_{IgL λ}}{{k}_{IgL κ}} = \frac{{N}_{{CT}_{IgL κ}}}{{N}_{{CT}_{IgL λ}}}$$

where the relative sensitivity K_{RS} reflects the difference in probes' fluorescence and binding efficiencies in the two assays. Inserting eq. 4 and 6 and rearranging we get (eq. 11):

$$\frac{{N}_{{0}_{IgL κ}}}{{N}_{{0}_{IgL λ}}} = {K}_{RS} * \frac{{( 1 + {E}_{IgL λ} )}^{{CT}_{IgL λ}}}{{( 1 + {E}_{IgL κ} )}^{{CT}_{IgL κ}}}$$

This is the central equation to calculate the ratio between the numbers of copies of two cDNA molecules. CT_{IgLκ} and CT_{IgLλ} are the CT values obtained from the PCR amplifications of the IgLκ and IgLλ cDNAs, E_{IgLκ} and E_{IgLλ} are the efficiencies of the two PCR equations determined as slopes in plots of CT vs. log N₀ in the serial dilutions of the samples, and K_{RS} is the relative sensitivity constant of the two PCR assays determined using test samples with known cDNA concentrations.

The fractions of IgLκ and IgLλ mRNA expressed as percentage are finally calculated as (eq. 12, and eq. 13):

$$IgL κ = 100 * \frac{{K}_{RS} * \frac{{( 1 + {E}_{IgL λ} )}^{{CT}_{IgL λ}}}{{( 1 + {E}_{IgL κ} )}^{{CT}_{IgL} κ}}}{1 + {K}_{RS} * \frac{{( 1 + {E}_{IgL λ} )}^{{CT}_{IgL λ}}}{{( 1 + {E}_{IgL κ} )}^{{CT}_{IgL κ}}}}$$

$$IgL λ = 100 * \frac{1}{1 + {K}_{RS} * \frac{{( 1 + {E}_{IgL λ} )}^{{CT}_{IgL λ}}}{{( 1 + {E}_{IgL κ} )}^{{CT}_{IgL κ}}}}$$

To determine PCR efficiencies in a biological sample by studying the effect of dilution on CT, the experimental variation in CT due to experimental uncertainty and variation in PCR efficiency owing to added components must be small compared to that caused by dilution. We established this to be the case by determining the experimental reproducibility using a typical patient sample that was analyzed for expression of the immunoglobulin kappa and lambda light chain in example 1. The PCR efficiencies in the biological samples are according to this invention determined by first converting the mRNA to cDNA and then serially diluting the sample determining the CT values of both reactions after each dilution. A single dilution is sufficient to estimate PCR efficiency, but the more dilutions made the higher is the accuracy. However, too extensive dilutions should be avoided, because if the number of molecules gets too few stochastic errors may be introduced (Vogelstein B, Kinzler K W: Digital PCR. Proc Natl Acad Sci USA 1999, 96: 9236-9241; Peccoud J, Jacob C: Theoretical uncertainty of measurements using quantitative polymerase chain reaction. Biophys J 1996, 71: 101-108). In example 2 we diluted 64 times in three steps of four times, which changed CT sufficiently to make experimental errors negligible. We also used samples that contained at least 6500 molecules of each cDNA, corresponding to at least 100 cDNAs of each in the most diluted sample.

Application in Cancer Diagnostics

Cancer is tissue that grows uncontrolled. The cancer cells have lost control of their cell division mechanism and divide indefinitely. All cancer cells originate in a single cell that has gone awry. In this cell genes that should be silent are active, and it often also loses ability to express growth controlling genes or expresses aberrant or foreign genes. Since all cancer cells originate from the same cell they share genetic signature, which can be used to detect and diagnose the cancer.

Particular kinds of cancer are lymphomas, which are cancers of the lymphatic system. Like other cancers lymphomas occur when cells divide too much and too fast. Growth control is lost, and the lymphatic cells may overcrowd, invade, and destroy lymphoid tissues and metastasize (spread) to other organs. There are two general types of lymphomas: “Hodgkin's Disease” (named after Dr. Thomas Hodgkin, who first recognized it in 1832) and non-Hodgkin's lymphoma (NHLs). Non-Hodgkin's Lymphomas caused by malignant (cancerous) B-cell lymphocytes represent a large subset (about 85% in the US) of the known types of lymphoma (the other two subsets being T-cell lymphomas and lymphomas where the cell type is unknown).

The traditional way to diagnose lymphoma is to take a surgical biopsy and test it by immunocytochemistry, flow cytometry and cytogenic studies. These tests rely on cell-specific antibodies. As alternative a fine needle aspiration (FNA) biopsy could be taken. This uses a very thin, hollow needle that is attached to a syringe. The needle is inserted into the swollen lump. It is then pushed back and forth to free some cells, which are aspirated (drawn up) into the syringe. FNA can distinguish noncancerous conditions, like infections, from NHLs or other cancers. FNA also is useful for staging, or determining the extent, of disease, and for monitoring recurrence, or return of cancer. But, because of small sample sizes and lack of information about lymph node structure, FNA often is inadequate for the initial diagnosis of NHL using current immunologic methods. A great improvement would be a more sensitive method than those based on immunochemistry, for which material from FNA would be sufficient.

B-lymphocytes produce immunoglobulins having a heavy chain and either a kappa (IgLκ) or a lambda (IgLλ) light chain. Each B-lymphocyte decides early in its development which light chain to produce. In healthy humans about sixty per cent of the B-cells produce kappa chains and the rest produce lambda chains. Normal lymphoid tissues therefore contain a mixture of B-cells with a IgLκ:IgLλ ratio of about 60:40 (Levy R, Warnke R, Dorfman R F, Haimovich J: The monoclonality of human B-cell lymphomas. J Exp Med 1977, 154:1014-1028; Barandun S, Morell A, Skvaril F, Oberdorfer A: Deficiency of kappa- or lambda-type immunoglobulins. Blood 1976, 47:79-89). Lymphomas, like all malignant tumors, are clonal and arise from one transformed cell. Lymphoma tissues are dominated by the tumor cells and consequently the IgLκ:IgLλ ratio is changed. Kappa producing tumors result in a higher IgLκ:IgLλ ratio, while lambda producing tumors result in a lower ratio. Assuming that the translation efficiency and stability of the IgLκ and IgLλ mRNAs are similar, clonality may be detected by measuring the IgLκ:IgLλ expression ratio. In Example 3 we show how patient samples can be classified as NHL positive and NHL negative from the determined IgLκ:IgLλ expression ratio by the method invented here. The excellent accuracy is impressive in view of the very little amount of material needed for analysis. The 1000 to 100000 representative cells typically obtained in a fine needle aspiration biopsy are sufficient for at least 50 tests by the real-time PCR assay and detection of possible B-cell monoclonality in the specimen by the invention presented here.

Another possible application of the method invented here is to detect T cell clonality. Here instead markers will be variants of the T cell receptors

Still another application of the method invented here is to monitor progress of disease. Some cancers are caused by expression of unnatural proteins, such as the bcr-abl fusion protein in Chronic Myelogenous/Myeloid Leukemia (CML) patients. It is important to quantify the amount of bcr-abl fusion transcript for diagnosis, and it is even more important to monitor disease progress. Imatinib mesylate (Gleevec® also known STI571) is a molecule in clinical trials for treatment of CML patients and to optimize treatment it is desired to know how patients respond to the drug, which is measured as changes in bcr-abl expression. Since drug treatment may affect overall gene expression, the expression of bcr-abl is usually determined relative to a house-keeping gene such as GAPDH. In Example 4 we show that bcr-abl and GAPDH PCR efficiencies are inhibited to different degree in CML patient sample and, hence, the importance of taking this into account when determining expression ratios and effect of drug treatment.

Indeed any diagnosis based on determining gene expression levels are possible applications of the method invented here. It is not limited to determining the ratio of expression of two genes; some diseases may be characterized by a particular expression pattern of three or even more genes.

Another possible application of the method invented here is to measure the relative amount of various splicing variants of a gene, which may be of interest in diagnosis or prognosis. The PCR efficiencies of the various splicing variants, which in general differ in both lengths and sequence, may vary, and correction may be important to obtain an accurate measure.

Another possible application of the method invented here is to measure the relative activity of alternative promoters of genes. These are also likely to be amplified with different efficiencies that should be taken into account for proper diagnosis and prognosis.

### EXAMPLES

### Example 1

### Experimental Reproducibility

Surgical lymph node biopsies from previously untreated patients were transported from the operation theatre in ice water chilled boxes and handled in the laboratory within 30 minutes. Material for the study was rapidly frozen in dry ice/isopentane and stored at −70° C.

Parts of the tissues were fixed in formalin and used for routine diagnostic analysis. Diagnosis was reached by a combination of microscopic evaluation of histology, immunostaining of several markers including the kappa and lambda chains (IHC) and in some cases flow cytometry. The samples were classified as lymphadenitis or malignant lymphoma according to the R.E.A.L.-terminology (Harris N H, Jaffe E S, Stein H, Banks P M, Chan J K, Cleary M L, Delsol G, De Wolf-Petters C, Falini B, Gatter K C: A proposal from the International Lymphoma Study Group. Blood 1994, 84:1361-1392).

RNA was extracted using the Fast Prep System (FastRNA Green, Qbiogene). Ten μg of total RNA was mixed with 2 μg of pdT oligomers (Pharmacia) and incubated at 65° C. for 5 minutes. First strand cDNA synthesis was then performed by adding 0.05 M tris-HCl, pH 8.3, 0.075 M KCl, 3 mM MgCl₂, 0.01 M DTT, 10 U/ml M-MLV reverse transcriptase (Life Technologies), 0.05 U/ml RNA guard (Life Technologies) and 10 mM of each deoxyribonuleotide to a final volume of 20 ml and incubating the samples at 37° C. for one hour. The reaction was terminated by incubation at 65° C. for 5 minutes and samples were stored at −80° C.

Two homopyrimidine light-up probes, H—CCTTTTTCCC—NH₂ (IgLκLUP) and CCTCCTCTCT-NH2 (IgLλLUP), directed against PCR amplification products of the constant regions in the human immunoglobulin kappa (IgLκ) and lambda (IgLλ) light-chains respectively, were designed. Both probes are homopyrimidine sequences, which are known to exhibit very large signal enhancement upon target binding (Svanvik N, Nygren J, Westman G, Kubista M: Free-probe fluorescence of light-up probes. J Am Chem Soc 2001, 123:803-809). Both probes had the thiazole orange derivate, N-carboxypentyl-4-[(3′-methyl-1′,3′-benzothiazol-2′-yl)methylenyl]quinolinium iodide (TO—N-5-COOH), as label. They were synthesized by solid phase synthesis and purified twice by reverse phase HPLC as described (Svanvik N, Westman G, Wang D, Kubista M: Light-up probes: thiazole orange-conjugated peptide nucleic acid for detection of target nucleic acid in homogeneous solution. Anal Biochem 2000, 281:26-35). Probe concentrations were determined spectroscopically assuming molar absorptivities at 260 nm of 83,100 M⁻¹cm⁻¹ for IgLκLUP and 81,100 M⁻¹cm⁻¹ for IgLλLUP.⁷ The probes were designed to have melting temperatures (Tₘ) of 65-70° C., which is in between the annealing (T_{anneling}=55° C.) and elongation (T_{elongation}=74° C.) temperatures of the PCR:s.

PCR products were purified by QIAquick™ PCR purification kit (Qiagen) and their concentrations were determined spectroscopically assuming molar absorptivity at 260 nm of 13,200 M⁻¹cm⁻¹ per base pair. Primer (Medprobe Inc) concentrations were estimated assuming ε₂₆₀/10³=12.0n_{G}+7.1n_{C}+15.2n_{A}+8.4n_{T} M⁻¹cm⁻¹, where n_{X} is the total number of base x (Current Protocolos in Molecular Biology. Edited by Ausubel F M, Brent R, Kingstone R, Moore D D, Seidman J G, Smith J A, Struhl K. John Wiley & Sons, Inc. Canada, 2000, pp. A.3D.2)

PCR systems were designed for a 231 bp fragment of the human IgLκ (GenBank accession number AK024974) and a 223 bp fragment of the human IgLλ (GenBank accession number X51755) comprising the IgLκLUP and IgLλUP target sequences, respectively. Reaction conditions were optimized as described elsewhere (Kubista M, St [symbol] ahlberg A, Bar T: Light-up probe based real-time Q-PCR. Proceedings of SPIE, in Genomics and Proteomics Technologies, Raghavachari R, Tan W, Editors. Proceedings of SPIE 2001, 4264:53-58). IgLκ and IgLλ PCR:s both contained 75 mM Tris (pH 8.8), 20 mM (NH₄)₂SO₄, 0.1% Tween 20, 1 U of JumpStart™ Taq DNA polymerase (with antibody) (Sigma-Aldrich) and 200 ng/μL of BSA. Specific components for the IgLκ PCR were 5 mM MgCl₂, 0.2 mM deoxyribonuleotides (Sigma-Aldrich), 800 nM of each primer (MedProbe) and 800 nM IgLκLUP, and for the IgLλ PCR 3.5 mM MgCl₂, 0.4 mM deoxyribonuleotides, 600 nM of each primer and 600 nM IgLλLUP. Primer sequences were for IgLκ 5′-TGA GCA AAG CAG ACT ACG AGA-3′ (forward) (SEQ. ID. NO.1) and 5′-GGG GTG AGG TGA AAG ATG AG-3′ (reverse) (SEQ. ID. NO. 2), and for IgLλ 5′-GAG CCT GAC GCC TGA G-3′ (forward) (SEQ. ID. NO. 3) and 5′-ATT GAG GGT TTA TTG AGT GCA G-3′ (reverse) (SEQ. ID. NO. 4).

Real-time PCR was measured in a LightCycler (Roche Diagnostics) using the thermocycler program: 3 min pre-incubation at 95° C. followed by 50 cycles for 0 s at 95° C., 10 s at 55° C. and 11 s at 74° C. Fluorescence was monitored at the end of the annealing phase using 470 nm excitation and 530 nm emission (the LightCycler F1 channel). All amplification curves were baseline adjusted by subtracting the arithmetic average of the five lowest fluorescence read-out values in each sample (arithmetic baseline adjustment in the LightCycler software). The threshold was set to a value of 1.00, which was significantly above background noise, and the number of cycles required to reach this level, CT, was determined (Higuchi R, Fockler C, Dollinger G, Watson R: Kinetic PCR analysis: real-time monitoring of DNA amplification reactions. Biotechnology (N Y) 1993, 11:1026-1030).

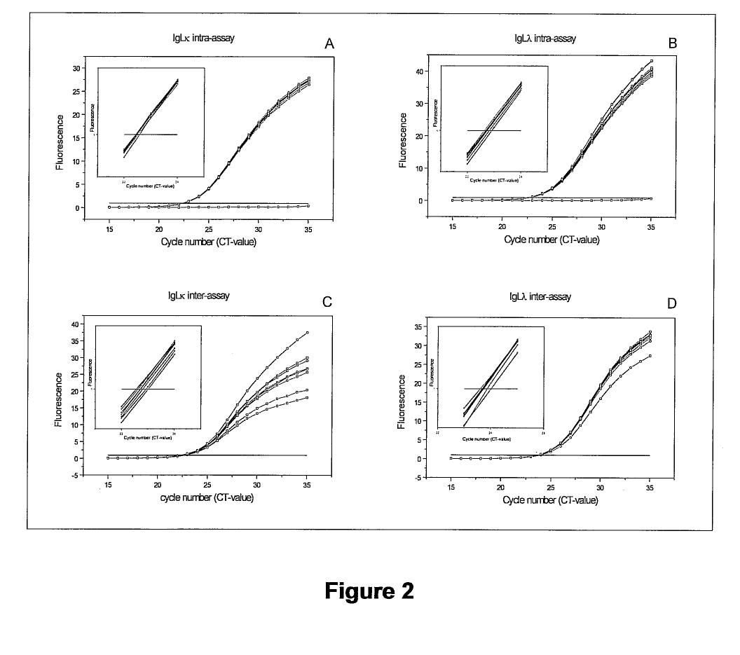

To classify a sample as either lymphoma negative with 60:40 IgLκ:IgLλ expression ratio or positive with a deviating expression ratio, we must know with what accuracy CT can be determined. We therefore designed experiments to measure the variation in CT due to experimental error and biological variability. First we studied the reproducibility of the PCR by splitting a sample into aliquots that were analyzed in parallel runs (intra-assay). We then also included variation due to sample handling by analyzing the same sample in independent runs (inter-assay). To minimize variation in template concentration between the two assays being compared a master mix containing template and all common PCR components was prepared and split into two aliquots to which the unique components for the IgLκ and the IgLλ reactions were added. Each experiment was performed 8 times using patient sample BR0 (FIG. 2).

In most reports PCR reproducibility is expressed as standard deviation in CT. The variance, SD², is (eq. 14)

$${SD}^{2} = \frac{{\sum }_{i = 1}^{n} {( {CT}_{i} - 〈 CT 〉 )}^{2}}{n - 1}$$

where <CT> is the average of the measured CT and standard deviation, SD, is the square root of the variance. However, since we are interested in determining the amount of cDNAs in the sample, the standard deviation of (1+E)^{-CT}, which is proportional to the number of cDNA molecules (eq. 1, and eq. 15):
N₀═N_{CT}*(1+E)^{-CT}

is more relevant. The variance in (1+E)^{-CT} is (eq. 16)

$${SD}^{2} = \frac{{\sum }_{i = 1}^{n} {( {( {( 1 + E )}^{- CT} )}_{i} - 〈 {( 1 + E )}^{- CT} 〉 )}^{2}}{n - 1}$$

where <(1+E)^{-CT}> is the average of (1+E)^{-CT}. To obtain the relative uncertainty in the number of cDNA molecules, we normalize the standard deviation with the average value to obtain the coefficient of variation, CV, which we express in percent (eq. 17):
CV=100×SD/<(1+E)^{-CT}>

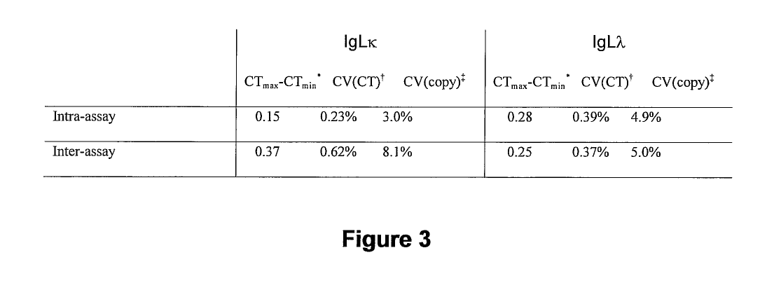

CV is the uncertainty in the determination of the number of cDNA molecules in the sample due to experimental factors. In the intra-assay, which reflects the reproducibility of the PCR, the coefficient of variation was 3.0% for the IgLκ reaction and 4.9% for the IgLλ reaction (FIG. 3). For the inter-assay, where also experimental errors contribute, the coefficients of variation were only slightly larger; 8.1% for the IgLκ reaction and 5.0% for the IgLλ reaction. Although it is not possible to calculate a coefficient of variation for the ratio of the two cDNAs we can estimate how much the IgLκ:IgLλ expression ratio in a negative sample could deviate from 60:40 due to experimental uncertainty in a bad case. Suppose the number of IgLκ cDNA is overestimated due experimental error by one standard deviation and the number of IgLλ cDNA is underestimated also by one standard deviation the measured ratio would be (60/40)×(1+0.081)/(1−0.050)=1.70=63/37. If instead the amount of IgLκ cDNA is underestimated and that of IgLλ cDNA is overestimated then the measured ratio would be (60/40)×(1−0.081)/(1+0.050)=1.31=56/44. Hence, due to experimental uncertainty and variation in PCR efficiency owing to added components we expect negative samples to display an IgLκ:IgL? expression ratio of 56:44<N₀_{_{IgLκ}}:N₀_{_{IgLλ}}<63:37.

### Example 2

### Determination of IgLκ and IgLλ PCR Efficiencies in Patient Samples

PCR efficiencies in seven patient samples were determined by diluting the test samples in steps and measuring CT value at each dilution. From these data intrinsic standard curves were constructed from which the PCR efficiencies are determined (FIG. 3). We chose to dilute the samples 64 times, in three steps of 4 times. The dilutions were performed in duplicates and the CT values were measured for both the IgLκ and IgLλ reactions determining the efficiencies of the two assays separately. Seven patient samples, four negative and three positive, were characterized this way, as well as purified template that should not contain any inhibitors.

The PCR efficiencies obtained when amplifying purified template were E_{IgLκ}=94.7% and E_{IgLλ}=93.2% signifying that both reactions proceed with very high efficiencies as expected for optimised PCR assays. Six of the patient samples exhibited efficiencies that were about 10% lower; the IgLλ PCR efficiency was 75.2%<E_{IgLλ}<85.8% with mean <E_{IgLλ}>=79.3% and the IgLκ efficiency was 79.4%<E_{IgLκ}<90.4% with mean <E_{IgLκ}>=85.4% (Table 2). The seventh sample, BR17, exhibited normal IgLκ efficiency (83.0%), while the IgLλ efficiency was only 58.9%. The reason for the extremely low efficiency of the IgLλ reaction in this sample is unclear. It was considered outlier and was not included in the calculation of average efficiencies.

When comparing the yields of two reactions the efficiency ratio (eq. 18)

$${X}_{ER} = \frac{( 1 + {E}_{IgL κ} )}{( 1 + {E}_{IgL λ} )}$$

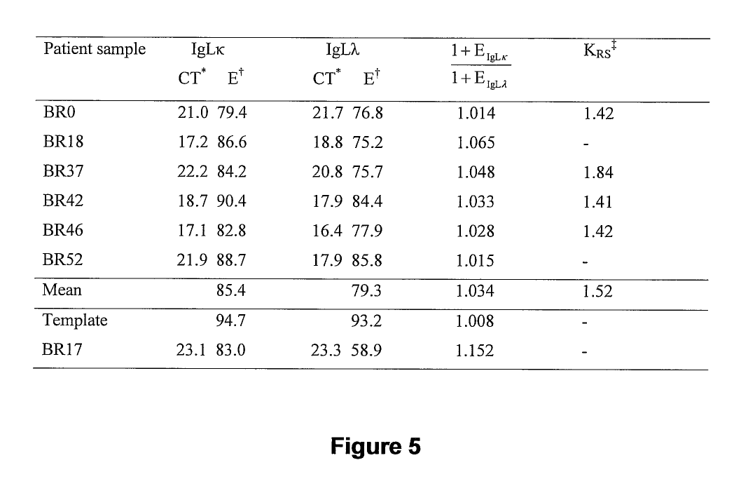

is the relevant parameter (see eq. 9). For the six samples 1.01<X_{ER}<1.065 with <X_{ER}>=1.034 (FIG. 5). Hence, after some 20 amplification cycles, which was typically required to reach threshold with the patient samples (FIG. 2), twice (1.034²⁰=2) as many kappa DNA molecules have been formed compared to lambda DNA due to the difference in PCR efficiencies.

Finally, to relate the measured CT-values of the two real-time PCR reactions to the ratio between the numbers of corresponding cDNA molecules, we must also determine the relative sensitivity, K_{RS}, of the two probing systems (eq. 8, and eq. 19).

$${K}_{RS} = \frac{{N}_{{0}_{IgL κ}}}{{N}_{{0}_{IgL λ}}} * \frac{{( 1 + {E}_{IgL κ} )}^{{CT}_{IgL κ}}}{{( 1 + {E}_{IgL λ} )}^{{CT}_{IgL λ}}}$$

was calculated from the CT values (CT_{IgLκ}, CT_{IgLλ}) and PCR efficiencies (E_{IgLκ}, E_{IgLλ}) determined for the four negative samples (table 2) assuming 60:40 IgLκ:IgLλ expression ratio. This gave 1.41≦K_{RS}≦1.84 with mean <K_{RS}>=1.52 (FIG. 5). As alternative K_{RS} was determined using purified template, which concentration was determined spectroscopically, that was diluted and amplified. Hence, the probing of IgLκ DNA is about 50% more sensitive than probing of IgLλ DNA using the probes and conditions here.

### Example 3

### Classification of NHL Lymphoma Patient Samples

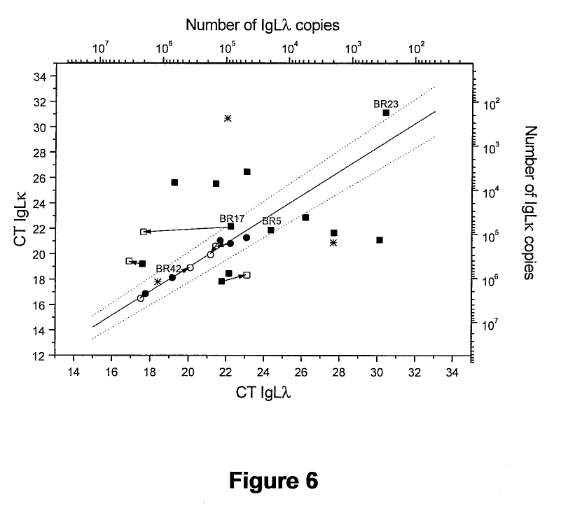

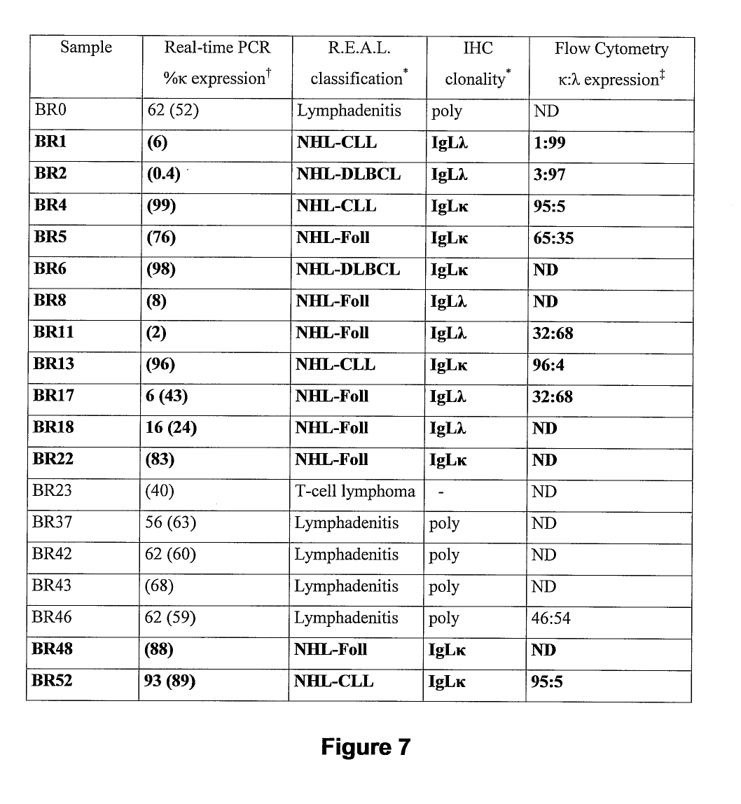

A total of 20 patient samples were analyzed for B-cell lymphoma by the Q-PCR assay. All samples were run in duplicates including negative controls. The data plotted in FIG. 6 and summarized in FIG. 7. In the plot each symbol represents one sample and is positioned on the coordinates CT_{IgLκ}, CT_{IgLλ}. The corresponding number of cDNA molecules of purified template, calculated assuming E_{IgLκ}=94.7% and E_{IgLλ}=93.2%, is indicated in logarithmic scale on the opposite axes. Samples considered negative by IHC analysis are shown as circles and positive samples are shown as squares.

Negative samples with IgLκ:IgLλ gene expression ratio of 60:40 are expected to lie on a straight line. Rewriting equation (9) gives (eq. 20):
N₀_{_{IgLκ}}*(1+E_{IgLκ})^{CT}^{_{IgLκ}}=K_{RS}*N₀_{_{IgLλ}}*(1+E_{IgLλ})^{CT}^{_{IgLλ}}

converting it to logarithmic form (eq. 21):

$${CT}_{IgL κ} * log ( 1 + {E}_{IgL κ} ) = log ( {K}_{RS} * \frac{{N}_{{0}_{IgL λ}}}{{N}_{{0}_{IgL κ}}} ) + {CT}_{IgL λ} * log ( 1 + {E}_{IgL λ} )$$

and rearranging, we obtain (eq. 22):

$${CT}_{IgL κ} = \frac{log ( 1 + {E}_{IgL λ} )}{log ( 1 + {E}_{IgL κ} )} * {CT}_{IgL λ} + \frac{log ( {K}_{RS} * \frac{{N}_{{0}_{IgL λ}}}{{N}_{{0}_{IgL κ}}} )}{log ( 1 + {E}_{IgL κ} )} = {CT}_{IgL κ} = k * {CT}_{IgL λ} + 1$$

This describes a linear relation between CT_{IgLκ} and CT_{IgLλ} with slope k and intercept l. Inserting <E_{IgLκ}>=0.854, <E_{IgLλ}>=0.793 and <K_{RS}>=1.52, which are the average values determined for the six samples above (FIG. 5), and N_{0IgLκ}/N_{0IgLλ}=60:40=1.5, we obtain k=0.946 and l=0.021. Note that the relative sensitivity, K_{RS}, was calculated from measurements on negative samples assuming 60:40 expression ratio (eq. 17). This cancels the N_{0IgLκ}/N_{0IgLλ} ratio in the nominator in the second term. Hence, the calculated slope and intercept of the relation between CT_{IgLκ} and CT_{IgLλ} for negative samples is independent of the assumption of a particular IgLκ:IgLλ expression ratio. A line with k=0.946 and l=0.021 is drawn in FIG. 6.

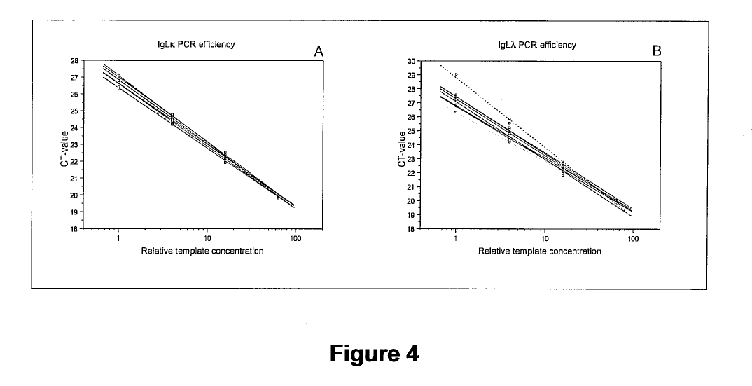

Some negative samples are slightly off the line representing 60:40 expression (FIG. 5). This may be due to variations in PCR efficiencies among the samples. Such variations will cause an error in the estimation of the number of cDNA molecules from the measured CT-values when mean PCR efficiencies are assumed. If the efficiencies of the two PCR assays in a sample deviate from the mean values to about the same degrees, the measured CT-values will still correctly reflect the expression ratio and negative samples will fall on the 60:40 line, although they will be displaced diagonally from where they would be if their efficiencies were normal. However, if the efficiency of one of the reactions deviates more than the other from the mean values, a negative sample may be off from the 60:40 line. For the seven samples characterized by the method invented here (FIG. 4, FIG. 5) the measured CT-values can be corrected for the differences between their specific PCR efficiencies and the mean efficiencies (eq. 23):

$${CT}_{corr} = {CT}_{meas} * \frac{log ( 1 + E )}{log ( 1 + 〈 E 〉 )}$$

The corrected CT-values are shown with open symbols and they are connected to the measured CT-values by arrows (FIG. 6). Although some arrows are diagonal, indicating that the two reactions are inhibited to about the same degree, which does not affect classification, there are some important exceptions.

To account for experimental error and variations in PCR efficiencies in classification of samples, we estimate limits within which negative samples should be found. Keeping the intercept fixed in eq. 20, gives (eq. 24):

$${CT}_{IgL κ} = \frac{log ( 1 + {E}_{IgL λ} )}{log ( 1 + {E}_{IgL κ} )} * {CT}_{IgL λ} + \frac{log ( {K}_{RS} * \frac{{N}_{{0}_{IgL λ}}}{{N}_{{0}_{IgL κ}}} )}{〈 log ( 1 + {E}_{IgL κ} ) 〉}$$

we calculate the standard deviation of the slope, k=log (1+E_{IgLλ})/log (1+E_{IgLκ}), from the efficiencies determined for the six samples (BR17 was excluded) characterized by in situ calibration. This gave SD=0.031. For a normal distribution 95% confidence interval is given by mean±1.96*SD. In FIG. 3 the dashed lines indicate the interval (eq. 25):
CT_{IgLκ}=(0.946±0.060)*CT_{IgLλ}+0.021

Although the confidence interval takes into account most of the experimental variation, it accounts neither for the variance in the intercept nor the natural variation in the IgLκ:IgLλ expression ratio among healthy individuals. These factors would broaden the confidence interval further. Hence, the interval indicates where negative samples are expected to be found with at least 95% probability. All negative samples in this study fall within this interval (FIG. 3).

Positive samples with IgLκ clonality are below the 60:40 line, while those with IgLλ clonality are above it. Most positive samples fall outside the confidence interval. However, there are some important exceptions. The most striking is BR17, which uncorrected falls within the confidence interval and would be classified as normal. However, after correction for its anomalous PCR efficiencies by the method invented here it falls far outside the confidence interval and can safely be classified as lymphoma with IgLλ clonality (FIGS. 6 and 7). The reason sample BR5 is within the interval was not established; most likely it is also due to anomalous PCR efficiencies. Sample BR23 has very high CT values, indicating very few copies of both IgLκ and IgLλ cDNA, and was found by IHC analysis to be a T-cell lymphoma.

### Example 4

### Determination of bcr-abl Transcription Relative to Transcription of GAPDH for CML Diagnosis in Patient Samples Using Taqman Based Real-Time PCR Assay

Peripheral blood samples from CML patients and controls were extracted at Sahlgrenska University hospital in Gothenburg, Sweden. White blood cells were counted and 100 000 cells were lysed in EL-buffer (Qiagen) and PBS, and stored at −20 until mRNA extraction. RNA-extraction was performed on the Genovision GenoM Robotic Workstation. PolydT coated magnetic beads were used to extract mRNA from lysed blood cells by applying a magnetic force separating the mRNA from other components. The other components are washed away and the mRNA can be eluted by heat. cDNA was synthesized in solution containing 1× Gibco buffer x5, 100 mM DDT, 1 mM dNTP, 20 μM random hexamers, 1 U/μl Rnase inhibitor, 10 U/μl Superscript II (Invitrogen). RNAse free water was added to a final volume of 50 μl to which 50 μl of mRNA from the extraction step was added. The resulting solution was run in a thermocycler at room temperature for 10 min, 42° C. for 50 min, 70° C. for 15 min, 95° for 5 min.

Primers used in the BCR-ABL reaction were GCATTCCGCTGACCATCAATA (b2a2-s), TCCAACGAGCGGCTTCAC (b2a2-as) and CCACTGGATTAGCAGAGTTCAA (b3a2-s). The sequence specific probe used was FAM-CAGCGGCCAGTAGCATCTGCTTTGA-BHQ1

Primers used in the GAPDH reaction CAACTGGGACGACTGGAGA (GAPDH-s) and GAAGATGGTGATGGGATTTC (GAPDH-as) and FAM-CAAGCTTCCCGTTCTCAGCC-DQ or FAM-CAAGCTTCCCGTTCTCAGCC-BHQ1 was used as sequence specific probe.

Solutions containing 1× Platinum PCR Buffer (Invitrogen), 4 mM MgCl₂ 0.5 mM dNTP, 1.25 U Platinum Taq polymerase (Invitrogen), 0.833 μM b2a2-s primer, 0.833 μM b3a2-s primer, 0.833 μM b2a2-as primer, 0.833 μM BCR-ABL probe, and 5 μl template from reverse transcription to a total volume of 20 μl for the BCR-ABL reaction. The corresponding solution for the GAPDH reaction contained 1× Platinum PCR Buffer (Invitrogen), 4 mM MgCl₂ 0.5 mM dNTP, 1.25 U Platinum Taq polymerase (Invitrogen), 0.833 μM GAPDH-s primer, 0.833 μM GAPDH-as primer, 0.833 μM GAPDH probe, and 5 μl template from reverse transcription to a total volume of 20 μl.

Samples were run in the Rotorgene (Corbett Research) with fluorescence excitation at 470 nm and emission at 510 nm. Thermal cycling was programmed at 2min initial denaturation at 95° C. and 50-55 cycles of 95° C. for 30 s and 60° C. for 60 s.

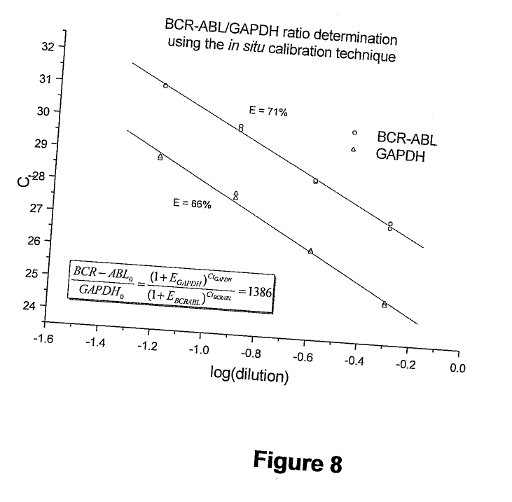

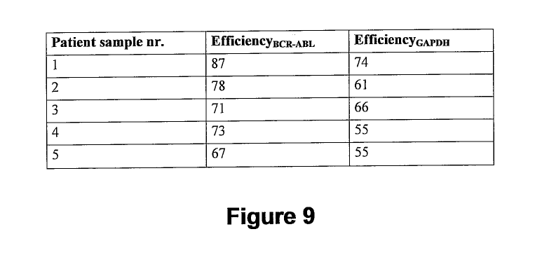

PCR efficiencies were determined by serially diluting the samples in four steps a two times (FIG. 8) for five patient samples (FIG. 9).

### Example 5

### Determination of bcr-abl and GAPDH Transcription Using Dye Assay

PCR-product template was prepared by amplification of BCR-ABL and GAPDH fragments in cDNA from K562 cells. The PCR-product was purified using the QIAquick PCR purification kit (Qiagen).

Primers used in the BCR-ABL reaction were GCATTCCGCTGACCATCAATA (b2a2-s), TCCAACGAGCGGCTTCAC (b2a2-as) and CCACTGGATTAGCAGAGTTCAA (b3a2-s). Primers used in the GAPDH reaction were CAACTGGGACGACTGGAGA (GAPDH-s) and GAAGATGGTGATGGGATTTC (GAPDH-as).

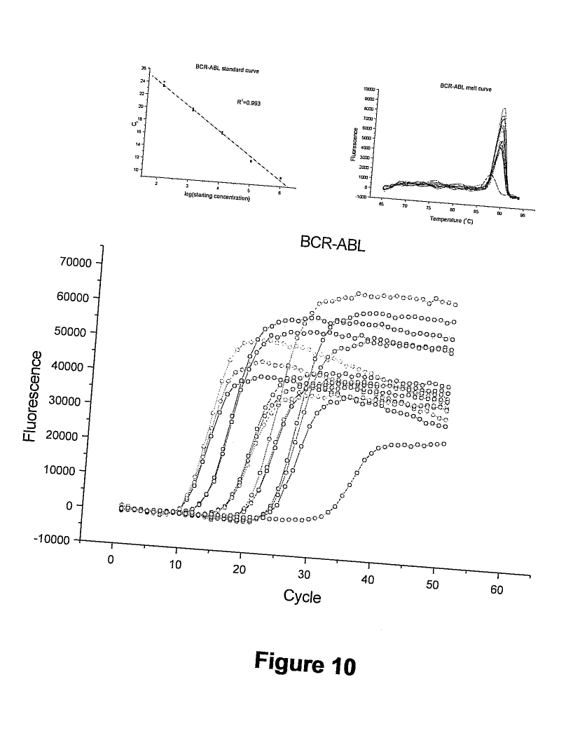

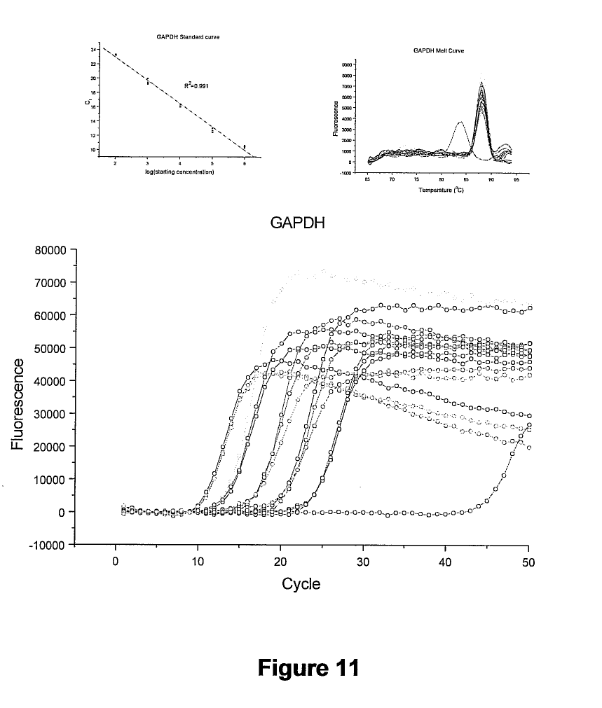

Solutions containing 1× Platinum PCR Buffer (Invitrogen), 4 mM MgCl₂ 0.5 mM dNTP, 1.25 U Platinum Taq polymerase (Invitrogen), 0.833 μM b2a2-s primer, 0.833 μM b3a2-s primer, 1:80 000 dilution of SYBR Green I, and 6.25 μl template from reverse transcription to a total volume of 25 μl for the BCR-ABL reaction (FIG. 10). The corresponding solution for the GAPDH reaction contained 1× Platinum PCR Buffer (Invitrogen), 4 mM MgCl₂ 0.5 mM dNTP, 1.25 U Platinum Taq polymerase (Invitrogen), 0.833 μM GAPDH-s primer, 0.833 μM GAPDH-as primer, 1:80 000 dilution of SYBR Green I, and 6.25 μl template from reverse transcription to a total volume of 25 μl (FIG. 11)

Samples were run in the iCycler (Bio-Rad) with fluorescence excitation at 490 nm and detection at 530 nm. Thermal cycling was programmed at 2 min initial denaturation at 95° C. and 50 cycles of 95° C. for 20 s, 60° C. for 20 s, 73° C. for 20 s. A melt curve was performed from 65° C. to 95° C.

Sequence Listing

SEQ. ID. NO. 1
Strand: Single
Nucleic acid
PCR primer
5′-TCT CGT AGT CTG CTT TGC TCA -3′
SEQ. ID. NO. 2
Strand: Single
Nucleic acid
PCR primer
5′-CT CAT CTT TCA CCT CAC CCC -3′,
SEQ. ID. NO. 3
Strand: Single
Nucleic acid
PCR primer
5′- C TCA GGC GTC AGG CTC -3′
SEQ. ID. NO. 4
Strand: Single
Nucleic acid
PCR primer
5′-C TGC ACT CAA TAA ACC CTC AAT -3′

1-20. (canceled)

21. A method for diagnosing and/or classifying a disease by comparing the expression ratio of two genes by determining the ratio of the corresponding mRNAs in a sample comprising the steps:
a. converting the mRNAs to cDNA and;
b. performing one or more controlled dilutions of the sample and;
c. amplifying each of the two genes in the sample, and in each said dilutions of the sample by PCR amplification while registering the number of amplification cycles required to obtain a preset signal threshold level (CT) and;
d. estimating the efficiency (E) of the PCR for each of the two genes in the particular sample from the dependence of CT on the logarithm of the dilution factor whereby the relative amounts of two genes present in a sample is determined using a relation:
$$\frac{{N}_{0 A}}{{N}_{0 B}} = {K}_{RS } \frac{{( 1 + {E}_{A} )}^{{CT}_{A}}}{{( 1 + {E}_{B} )}^{{CT}_{B}}}$$
wherein N₀ reflects the number of units, N, present at the time zero of cDNA of the two genes, and the relative sensitivity K_{RS} reflects a difference in the fluorescence and binding efficiencies of primers used in the two PCR assays.

22. The method according to claim 21, wherein the disease is lymphoma and the relative expression of the genes is different in malignant tissue compared to healthy tissue.

23. The method according to claim 22, wherein at least a pair of the genes, the expression of which is compared, are the immunoglobulin kappa and lambda light chains.

24. The method according to claim 22, wherein either of the two genes is expressed in each clone of lymphocytes, and are present in a particular ratio in healthy individuals, which ratio is altered in positive samples due to clonality indicating presence of lymphoma.

25. The method according to claim 24, wherein the expression of the immunoglobulin kappa and lambda light chains is compared by determining the IgLλ:IgL mRNA ratio by reverse transcription PCR.

26. The method according to claim 25, wherein the expression of the immunoglobulin kappa and lambda light chains is compared by determining IgLk:IgLλ mRNA ratio by real-time PCR.

27. The method according to claim 21, wherein one or more of PCR primers used for the assay of the gLk gene are complementary to
(SEQ. ID. NO. 1)
5′-TCT CGT AGT CTG CTT TGC TCA - 3′,
and
(SEQ. ID. NO. 2)
5′-CT CAR CTT TCA CCT CAC CCC - 3′,
respectively.

28. The method according to claim 21, wherein one or more of PCR primers used for the assay of the IgLλ gene are complementary to
(SEQ. ID. NO. 3)
5′- C TCA GGC GTC AGG CTC - 3′
and
(SEQ. ID. NO. 4)
5′-C TGC ACT CAA TAA ACC CTC AAT -3′,
respectively.

29. The method according to claim 21, wherein the disease Chronic Myelogenous/Myleoid Leukemia (CML) is diagnosed by determining the expression of bcr-abl fusion transcript relative to the GAPDH transcription.

30. The method according to claim 21, wherein the expression of three or more genes are compared.
